(19) United States
(12) Patent Application Publication
(10) Pub. No.: US 2025/0042793 A1
(43) Pub. Date: Feb. 6, 2025

(54) MANGANESE CARBONATE-SUPPORTED FERRIHYDRITE MATERIAL, AND PREPARATION AND USE THEREOF

(71) Applicant: QINGDAO UNIVERSITY OF TECHNOLOGY, Qingdao (CN)

(72) Inventors: Huawei WANG, Qingdao (CN); Rongxue ZOU, Qingdao (CN); Zhibo YANG, Qingdao (CN); Yanan WANG, Qingdao (CN); Xueqin WANG, Qingdao (CN); Yingjie SUN, Qingdao (CN); Yujun WANG, Qingdao (CN); Shupeng LI, Qingdao (CN)

(21) Appl. No.: 18/920,837

(22) Filed: Oct. 18, 2024

(30) Foreign Application Priority Data
Jun. 14, 2024 (CN) 202410766037.9

(51) Int. Cl. C02F 3/34; C01G 45/00; C02F 3/10; C02F 101/10; C12N 1/20

(52) U.S. Cl. CPC C02F 3/348; C01G 45/00; C02F 3/107; C12N 1/20; C02F 2101/103

(57) ABSTRACT

A manganese carbonate-supported ferrihydrite material preparation method, in which manganese sulfate and ammonium bicarbonate are respectively dissolved to obtain a manganese sulfate solution and an ammonium bicarbonate solution, and the manganese sulfate solution is added with ferrihydrite, dropwise added with the ammonium bicarbonate solution and reacted under magnetic stirring to obtain a precipitate. The precipitate is washed with distilled water and dried in a drying oven to obtain the manganese carbonate-supported ferrihydrite material. A method for treating wastewater polluted by arsenic (As) and antibiotics by using the manganese carbonate-supported ferrihydrite material is also provided.

### CROSS-REFERENCE TO RELATED APPLICATIONS

This application claims the benefit of priority from Chinese Patent Application No. 202410766037.9, filed on Jun. 14, 2024. The content of the aforementioned application, including any intervening amendments thereto, is incorporated herein by reference in its entirety.

### TECHNICAL FIELD

This application relates to wastewater treatment technologies, and more particularly to a manganese carbonate-supported ferrihydrite material, and a preparation and use thereof.

### BACKGROUND

The continuous growth of global population and rapid development of industry and agriculture, medical industry, livestock and aquaculture are accompanied by the serious water pollution associated with metalloid arsenic (As) and tetracyclines (TC) in recent years. TC has been frequently detected in a variety of environmental samples, and usually exists at ng/L or μg/L level in the sewage treatment plant effluent, surface runoff and groundwater. Some antibiotics and heavy metals are often added as a growth promoter to the animal feed, however, the antibiotics and heavy metals are difficult to be digested and absorbed, and are often excreted through animal urine and feces, which can enter the water environment through the surface runoff, causing risks of ecology and health. Considering the strong toxicity of As and TC, they will pose a serious threat to human health, therefore, it is urgent to remediate water pollution associated with As and TC.

At present, the wastewater polluted by As and antibiotics is remedied mainly by adsorption, flocculation, membrane separation or a zero-valent iron-based method, where the adsorption method is frequently used. Arsenic ions can interact with a variety of functional groups in the antibiotic to form different complexes, thereby affecting the adsorption effects of adsorbents. The adsorbents used in the adsorption method have wide sources, low cost, simple operation and strong adsorption ability, and have removal effects on a variety of antibiotics and metals. However, the adsorption method also has some inherent limitations, for example, most of the adsorption materials are non-regeneration, and have a limited applicable pH range, and the pollutants cannot be essentially degraded by adsorption. Unlike the adsorption method, the flocculation method involves the formation of large flocs from colloidal particles in the presence of some polymeric flocculants based on the bridging action. The flocculation method has advantages of simple operation, low cost and high efficiency. However, the traditional flocculants cannot trigger the flocculation of variably charged combined pollutants, and it is needed to modify the existing flocculants to realize efficient treatment of the As-antibiotic compound wastewater. The membrane separation method utilizes membrane materials with selective separation function to realize separation and purification of different components in the wastewater. The membrane separation method has advantages of simple process, strong functional selectivity and low energy consumption, and can be combined with other processing technologies. However, after absorbing pollutants in wastewater, the membranes used in the membrane separation method cannot be fully recycled, resulting in low utilization efficiency. Therefore, it is of important environmental significance to develop an efficient, economical and green remediation technology for the wastewater polluted by As and antibiotics.

### SUMMARY

An object of this application is to provide a manganese carbonate-supported ferrihydrite material, and a preparation and use thereof. This application has simple operation and strong adjustability. The manganese carbonate-supported ferrihydrite material, as a solid-phase manganese source, can accelerate the dissolution of manganese(II) (Mn(II)) and promote the formation of biogenic manganese oxides (BMO), thereby effectively improving the remediation effect of wastewater polluted by arsenic (As) and antibiotics. Therefore, this application has important environmental significance.

Objects of this application are achieved through the following technical solutions.

In a first aspect, this application provides a method for preparing a manganese carbonate-supported ferrihydrite material, comprising:
(S1) dissolving manganese sulfate with water to obtain a manganese sulfate solution, and dissolving ammonium bicarbonate with water to obtain an ammonium bicarbonate solution;
(S2) adding ferrihydrite and dropwise adding the ammonium bicarbonate solution to the manganese sulfate solution followed by reaction under magnetic stirring to obtain a first precipitate; and
(S3) washing the first precipitate with distilled water followed by drying in a drying oven to obtain the manganese carbonate-supported ferrihydrite material.

In some embodiments, in step (S1), a concentration of the manganese sulfate solution is 0.5-1.2 mol/L; and a concentration of the ammonium bicarbonate solution is 1.5-2.3 mol/L.

In some embodiments, in step (S2), an addition amount of the ferrihydrite is 50-120 g/L.

In some embodiments, in the step (S2), the ferrihydrite is prepared through steps of:
(S2.1) dissolving 40 g of ferric nitrate nonahydrate in 500 mL of water followed by magnetic stirring to obtain a ferric nitrate solution;
(S2.2) adding 310 mL of a 1 mol/L of potassium hydroxide solution to the ferric nitrate solution at a speed of 100 mL/min to obtain a mixed solution;
(S2.3) adjusting the mixed solution to a pH of 7-8 with the 1 mol/L of potassium hydroxide solution followed by stirring for 1 h to produce a supernatant and a second precipitate; and
(S2.4) washing the second precipitate with a 0.1 mol/L of sodium chloride solution 2-3 times followed by centrifugation at 3000 r/min for 8-12 min, and then drying at 80° C. for 10-12 h to obtain the ferrihydrite.

In some embodiment, in step (S3), the first precipitate is washed 2-4 times successively with 90-100° C. distilled water and 20-30° C. distilled water; and the drying is performed at 80-90° C. in the drying oven for 14-20 h.

In a second aspect, this application provides the manganese carbonate-supported ferrihydrite material prepared by the above preparation method.

In a third aspect, this application provides a method for treating a wastewater sample polluted by arsenic (As) and an antibiotic by using the manganese carbonate-supported ferrihydrite material, comprising:
(a) inoculating a Pseudomonas putida strain MnB1 with an accession number of ATCC 23483 into a culture medium A for culture to obtain a Pseudomonas putida suspension; and
(b) adding the Pseudomonas putida suspension into a culture medium B containing the manganese carbonate-supported ferrihydrite material followed by adding of 4-(2-hydroxyethyl) piperazine-1-ethanesulfonic acid (HEPES) in a clean bench to realize removal of As and the antibiotic.

In some embodiments, in step (a), the culture medium A comprises 0.3-0.8 g/L of a yeast extract, 0.2-0.8 g/L of casein acid hydrolysate, 0.3-0.8 g/L of glucose, 0.1-0.4 g/L of calcium chloride, 0.1-0.6 g/L of magnesium sulfate, 1-5 mL/L of a trace element and 1-3 mL/L of ferric chloride, and a pH of the culture medium A is 7; and the culture is performed through steps of:
inoculating the Pseudomonas putida strain MnB1 into the culture medium A with an inoculation amount of 3-5% by volume followed by culture under shaking and aerobic conditions at 25-35° C. for 1-2 days.

In some embodiments, in step (b), an addition amount of the Pseudomonas putida suspension is 3-5% by volume of the culture medium B; an addition amount of the manganese carbonate-supported ferrihydrite material is 0.1-2.0 g/L; the culture medium B comprises 0.1-0.4 g/L of ammonium ferrous sulfate, 0.1-0.4 g/L of sodium citrate, 0.05-0.2 g/L of yeast extract powder and 0.02-0.1 g/L of sodium pyrophosphate, and a pH of the culture medium B is 6-8; an initial concentration of As is 0.5-3 mg/L; and an initial concentration of the antibiotic is 10-40 mg/L.

The present disclosure has the following beneficial effects.
(1) The ferrihydrite has a large specific surface area and strong absorbability, and shows strong adsorption and complexation of arsenic. An adsorption and fixation efficiency of As(V) can be improved by constructing a supported manganese carbonate on the ferrihydrite matrix.
(2) The present disclosure has simple operation and strong adjustability. The manganese carbonate-supported ferrihydrite material, as the solid-phase manganese source, can accelerate the dissolution of Mn(II) and promote the formation of the BMO, thereby effectively improving the remediation effect of wastewater containing arsenic and antibiotics.
(3) In the wastewater treatment strategy provided herein, the bacterial growth and reproduction promote the dissolution of manganese carbonate (MnCO₃) to produce free Mn(II) ions, which will be oxidized into Mn(III) by an enzymatic reaction or under the action of a superoxide free radical; the Mn(III) undergoes a disproportionation reaction to produce the BMO, which can quickly oxidize As(III) to As(V); at the same time, the ferrihydrite plays a role as bacterial carrier to help manganese oxidizing bacteria dissolve the manganese carbonate to promote the oxidation of Mn(II) ions, and also provides more binding sites for As(V), which is conducive to the removal of As(V). The As removal process is accompanied by the BMO consumption and release of the Mn(II) ions, and the produced Mn(II) ions can be re-oxidized under the bacterial action to form the BMO or be adsorbed on the BMO surface. In addition to oxidizing the As(III), the BMO can also quickly oxidize and degrade TC. The TC can be degraded into small-molecule organic compounds through multi-step oxidation, and is finally completely mineralized and transformed into carbon dioxide (CO₂) and water. Through the above steps, the wastewater containing arsenic and antibiotics can be effectively remedied.

### BRIEF DESCRIPTION OF THE DRAWINGS

FIG. 1a is a scanning electron microscopy (SEM) image of manganese carbonate (MnCO₃).

FIG. 1b is an energy dispersive spectrometry (EDS) image of MnCO₃.

FIG. 1c is a SEM image of a manganese carbonate-supported ferrihydrite material (MnCO₃Fe) according to an embodiment of the present disclosure.

FIG. 1d is an EDS image of the MnCO₃Fe according to an embodiment of the present disclosure.

FIGS. 2a-d respectively show change curves of manganese(II) (Mn(II)) concentration, biogenic manganese oxide (BMO) concentration, arsenic (As) removal rate and tetracyclines (TC) removal rate over time in Example 1 of the present disclosure.

FIGS. 3a-d respectively show change curves of Mn(II) concentration, BMO concentration, As removal rate and TC removal rate over time in Example 2 of the present disclosure.

FIGS. 4a-d respectively show change curves of Mn(II) concentration, BMO concentration, As removal rate and TC removal rate over time in Example 3 of the present disclosure.

FIGS. 5a-d respectively show change curves of Mn(II) concentration, BMO concentration, As removal rate and TC removal rate over time in Example 4 of the present disclosure.

FIGS. 6a-d respectively show change curves of Mn(II) concentration, BMO concentration, As removal rate and TC removal rate over time in Example 5 of the present disclosure.

FIGS. 7a-d respectively show change curves of Mn(II) concentration, BMO concentration, As removal rate and TC removal rate over time in Example 6 of the present disclosure.

### DETAILED DESCRIPTION OF EMBODIMENTS

This application provides a method for removing arsenic (As) and antibiotics from wastewater, which is specifically performed as follows.

### (S1) Preparation of Manganese Carbonate-Supported Ferrihydrite Material (MnCO₃Fe)

Manganese sulfate and ammonium bicarbonate are respectively dissolved to obtain a 0.5-1.2 mol/L manganese sulfate solution and a 1.5-2.3 mol/L ammonium bicarbonate solution. The manganese sulfate solution is added with 50-120 g/L of ferrihydrite, dropwise added with the ammonium bicarbonate solution and reacted under magnetic stirring to obtain a first precipitate. The first precipitate is washed 2-4 times sequentially with 90-100° C. distilled water and 20-30° C. distilled water and dried in a drying oven at 80-90° C. for 14-20 h to obtain the manganese carbonate-supported ferrihydrite material.

The ferrihydrite is prepared through the following steps. 40 g of ferric nitrate nonahydrate is dissolved in 500 mL of water and magnetically stirred. The resultant ferric nitrate solution is added with 310 mL of a 1 mol/L potassium hydroxide solution at a speed of 100 mL/min, adjusted to pH 7-8 with the 1 mol/L potassium hydroxide solution, and vigorously stirred for 1 h to produce a supernatant and a second precipitate. The supernatant is discarded, and the second precipitate is washed 2-3 times with a 0.1 mol/L sodium chloride solution, centrifuged at 3000 r/min for 8-12 min, and then dried at 80° C. for 10-12 h to obtain the ferrihydrite.

(S2) Enrichment Culture of Pseudomonas putida Strain MnB1

Pseudomonas putida strain MnB1 is inoculated into a culture medium A with an inoculation amount of 3-5% by volume and cultured under horizontal shaking (150-180 rpm) and aerobic conditions at 25-35° C. for 1-2 days to obtain a Pseudomonas putida suspension.

The Pseudomonas putida strain MnB1, a manganese oxidizing typical strain, is purchased from the American Type Culture Collection (ATCC) with an accession number of ATCC 23483. The culture medium A includes 0.3-0.8 g/L of a yeast extract, 0.2-0.8 g/L of casein acid hydrolysate, 0.3-0.8 g/L of glucose, 0.1-0.4 g/L of calcium chloride, 0.1-0.6 g/L of magnesium sulfate, 1-5 mL/L of a trace element and 1-3 mL/L of ferric chloride, and a pH of the culture medium A is 7.

### (S3) Remediation Process

The Pseudomonas putida suspension obtained in step (S2) is inoculated into a culture medium B containing the manganese carbonate-supported ferrihydrite material, to which 4-(2-hydroxyethyl) piperazine-1-ethanesulfonic acid (HEPES) is added in a clean bench. MnCO₃ and MnCO₃Fe are respectively used as a solid-phase manganese source to investigate removal effects of As and TC through in-situ formation of BMO under the induction of a manganese oxidizing strain. As(III) with an initial concentration of 0.5-3 mg/L and TC with an initial concentration of 10-40 mg/L are employed as pollutants. The culture medium B is cultured under horizontal shaking at 20-35° C. and 150-180 rpm for 13-15 days. Several factors, including As(III) concentration, TC concentration and the addition amount of the MnCO₃Fe, have been taken into consideration to investigate the influence on the removal effects of As and TC.

In step (S3), an addition amount of the manganese carbonate-supported ferrihydrite material is 0.1-2.0 g/L. The Pseudomonas putida suspension is 3-5% by volume of the culture medium B. The culture medium B includes 0.1-0.4 g/L of ammonium ferrous sulfate, 0.1-0.4 g/L of sodium citrate, 0.05-0.2 g/L of yeast extract powder and 0.02-0.1 g/L of sodium pyrophosphate, and a pH of the culture medium B is 6-8.

### Example 1

A method for removing As and antibiotics from water was provided, which included the following steps.

(S1) 4.23 g of manganese sulfate was dissolved in 25 mL of deionized water to obtain a manganese sulfate solution, and 3.95 g of ammonium bicarbonate was dissolved in 25 mL of deionized water to obtain an ammonium bicarbonate solution. The manganese sulfate solution was added with 2.87 g of ferrihydrite, dropwise added with the ammonium bicarbonate solution at 5 mL/min until complete reaction under magnetic stirring to obtain a first precipitate. The first precipitate was washed 3 times sequentially with 100° C. distilled water and 30° C. distilled water, and dried in a drying oven at 80° C. for 14 h to obtain the manganese carbonate-supported ferrihydrite material (MnCO₃Fe).

The ferrihydrite is prepared through the following steps. 40 g of ferric nitrate nonahydrate was dissolved in 500 mL of water and magnetically stirred. The resultant ferric nitrate solution was added with 310 mL of a 1 mol/L of potassium hydroxide solution at a speed of 100 mL/min, adjusted to pH 7-8 with the 1 mol/L of potassium hydroxide solution, and vigorously stirred for 1 h to produce a supernatant and a second precipitate. The second precipitate was washed 2-3 times with a 0.1 mol/L of sodium chloride solution, centrifugated at 3000 r/min for 8-12 min, and then was dried at 80° C. for 10-12 h to obtain the ferrihydrite.

(S2) Pseudomonas putida strain MnB1 was inoculated into a culture medium A with an inoculation amount of 3% by volume and cultured under horizontal shaking (150 rpm) and aerobic conditions at 30° C. for 2 days to obtain a 1×10⁷ cfu/mL of Pseudomonas putida suspension.

In step (S2), the culture medium A included 0.5 g/L of a yeast extract, 0.5 g/L of casein acid hydrolysate, 0.5 g/L of glucose, 0.29 g/L of calcium chloride, 0.5 g/L of magnesium sulfate, 1 mL/L of a trace element solution ingredients (10 mg/L of copper sulfate pentahydrate (CuSO₄·5H₂O), 44 mg/L of zinc sulfate heptahydrate (ZnSO₄·7H₂O), 20 mg/L of cobalt chloride hexahydrate (CoCl₂·6H₂O) and 13 mg/L of sodium molybdate dihydrate (Na₂MoO₄·2H₂O) were dissolved in deionized water) and 1 mL/L of ferric chloride, and a pH of the culture medium A is 7. The Pseudomonas putida strain MnB1 selected herein was purchased from American Type Culture Collection (ATCC) with an accession number of ATCC 23483.

(S3) The manganese carbonate-supported ferrihydrite material prepared in step (S1) was added into a culture medium B, and a concentration of the manganese carbonate-supported ferrihydrite material was 0.5 g/L.

(S4) 3% by volume of the Pseudomonas putida suspension obtained in step (S2) was inoculated into the culture medium B containing 0.5 g/L of the manganese carbonate-supported ferrihydrite material.

(S5) HEPES (as a buffer to maintain a system pH) with a final concentration of 20 mmol/L was added into the culture medium B. As(III) with an initial concentration of 2 mg/L and TC with an initial concentration of 20 mg/L were added into the culture medium B as pollutants. The culture medium B was cultured under horizontal shaking (150 rpm) and aerobic conditions at 30° C. for 15 days. Mn(II) concentration, BMO concentration, As concentration and TC concentration were regularly monitored, and a control treatment was performed by replacing the manganese carbonate-supported ferrihydrite material with manganese carbonate.

In step (S5), the manganese carbonate was prepared through the following steps. 4.23 g of manganese sulfate was dissolved in 25 mL of deionized water to obtain a manganese sulfate solution, and 3.95 g of ammonium bicarbonate was dissolved in 25 mL of deionized water to obtain an ammonium bicarbonate solution. The manganese sulfate solution was dropwise added with the ammonium bicarbonate solution at a speed of 5 mL/min at room temperature under magnetic stirring to obtain a third precipitate. The third precipitate was washed 3 times sequentially with 100° C. distilled water and 30° C. distilled water and dried in the drying oven at 80° C. for 14 h to obtain the manganese carbonate (MnCO₃).

### Example 2

A method provided herein for removing combined pollution of As and antibiotics from wastewater was basically the same as Example 1, and the difference was that a concentration of manganese carbonate-supported ferrihydrite material was 1 g/L.

### Example 3

A method provided herein for removing combined pollution of As and antibiotics from wastewater was basically the same as Example 1, and the difference was that an initial concentration of As(III) was 0.5 mg/L.

### Example 4

A method provided herein for removing combined pollution of As and antibiotics from wastewater was basically the same as Example 1, and the difference was that an initial concentration of As(III) was 1 mg/L.

### Example 5

A method provided herein for removing combined pollution of As and antibiotics from wastewater was basically the same as Example 1, and the difference was that an initial concentration of TC was 10 mg/L.

### Example 6

A method provided herein for removing combined pollution of As and antibiotics from wastewater was basically the same as Example 1, and the difference was that an initial concentration of TC was 30 mg/L.

### Test Example 1

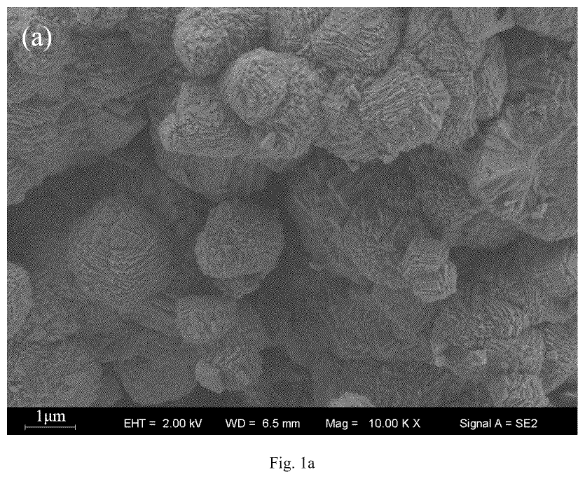

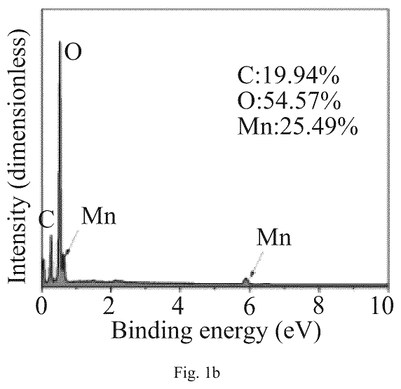

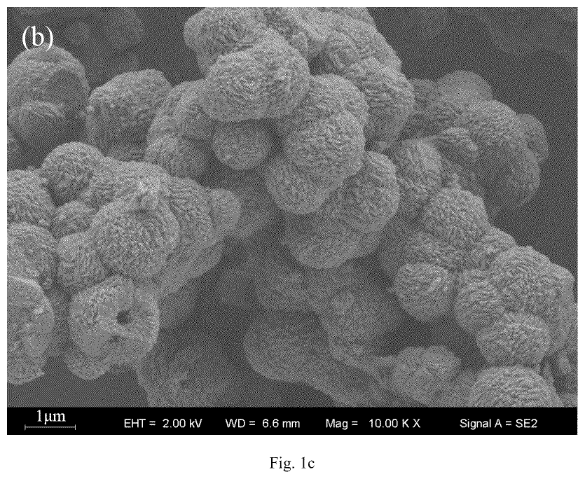

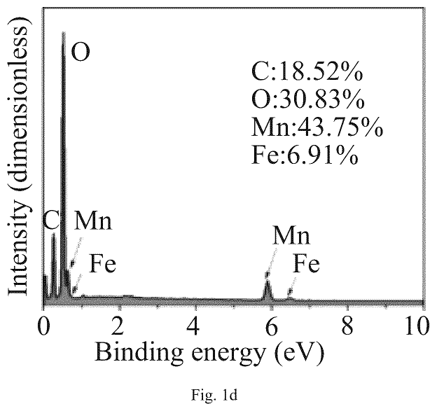

A Zeiss Super 55 VP scanning electron microscopy (SEM) was adopted to detect the MnCO₃Fe and MnCO₃ obtained in Example 1. SEM images and energy dispersive spectrometry (EDS) images were shown in FIGS. 1a-d. Both MnCO₃ and MnCO₃Fe were closely connected spheroids with same size arrangements and rough surfaces, and had dentate structures, and no significant difference were observed between appearances of MnCO₃Fe and MnCO₃. The samples were semi-quantitatively analyzed by EDS to obtain relative contents of carbon (C), oxygen (O), Mn and iron (Fe). Relative contents of C, O and Mn in MnCO₃ were respectively 19.94%, 54.57% and 25.49%, and relative contents of C, O, Mn and Fe in MnCO₃Fe were respectively 18.52%, 30.83%, 43.75% and 6.91%, showing that the content of Fe in MnCO₃Fe was significantly increased.

### Test Example 2

Mn(II) concentration, BMO concentration, As concentration and TC concentration of solutions in Examples 1-6 were measured.

Mn(II) concentration was measured with a formaldoxime-spectrophotometry. Under alkaline conditions, Mn(II) was oxidized to Mn(III), and Mn(III) reacted with formaldoxime to produce a brown complex with a maximum absorption peak at 450 nm.

BMO concentration was measured with a leucoberbelin blue (LBB) chromogenic method. A LBB solution was colorless and would quickly be turned into bright blue after reaction with manganese oxides. An absorption wavelength of the LBB chromogenic method was 620 nm.

As concentration was measured with an atomic fluorescence spectrophotometer (AFS-830) from Beijing Jitian Instrument Co., LTD. Conditions of the AFS-830 were as follows: 280 V of negative high voltage; 70 mA of lamp current; 200° C. of a preheating temperature of an atomizer; 600 mL/min of carrier gas flow; 800 ml/min of shield gas flow; and 0-20 μg/L of a standard curve concentration of As.

TC concentration was measured with a high-performance liquid chromatograph from Hitachi, Ltd. with octadecylsilane-C18 (ODS-C18) columns. Conditions of the high-performance liquid chromatograph were as follows: a mobile phase of 0.01 mol/L of dipotassium hydrogenphosphate solution (pH of 2.5) and acetonitrile with a volume ratio of 65:35; 25° C. of a column temperature; 360 nm of detection wavelength; 0.8 mL/min of flow rate; 20 μL of an injection volume; and 4.05 min of residence time of TC. Quantitative analyses of TC were carried out by an external standard method.

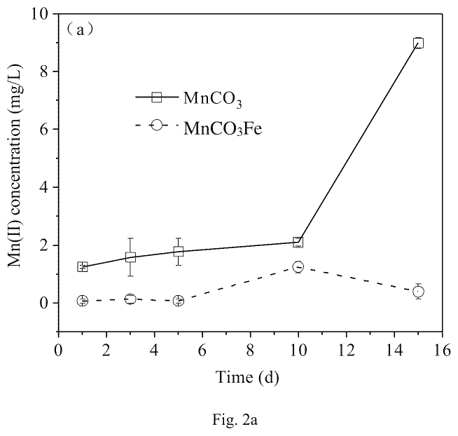

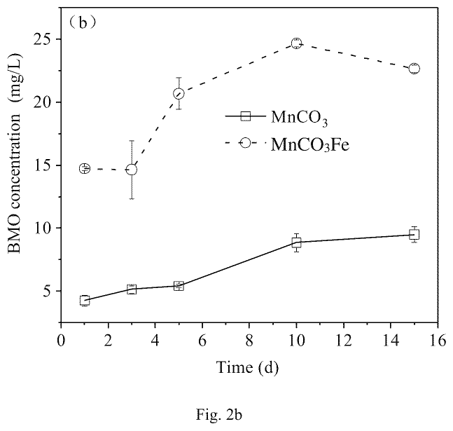

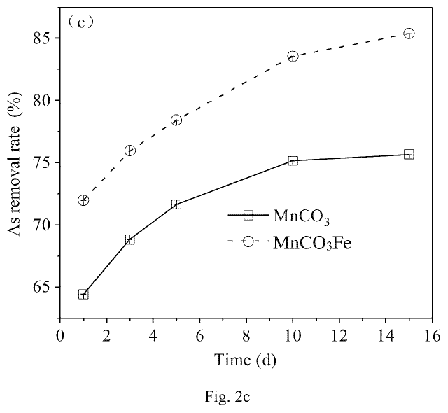

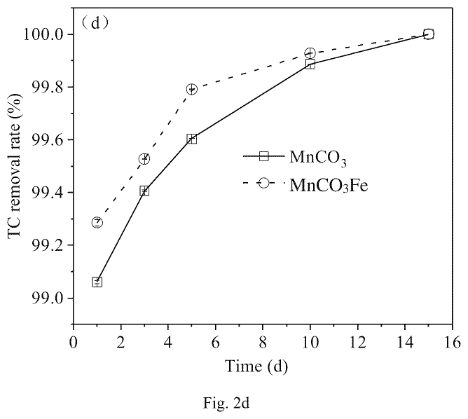

In Example 1, change curves of Mn(II) concentration, BMO concentration, As removal rate and TC removal rate over time were shown in FIGS. 2a-d. Referring to FIGS. 2a-d, a combined pollution system of As and the antibiotic were remediated under conditions in Example 1, and a concentration of soluble Mn(II) of MnCO₃Fe was less than 10 mg/L, and was less than that when the solid-phase manganese source was MnCO₃. When the solid-phase manganese source was MnCO₃Fe, a concentration of BMO significantly increased, a total As removal rate reached 85.37%, and a total TC removal rate reached 100%. When the solid-phase manganese source was MnCO₃, the total As removal rate reached 75.66%, and the total TC removal rate reached 100%.

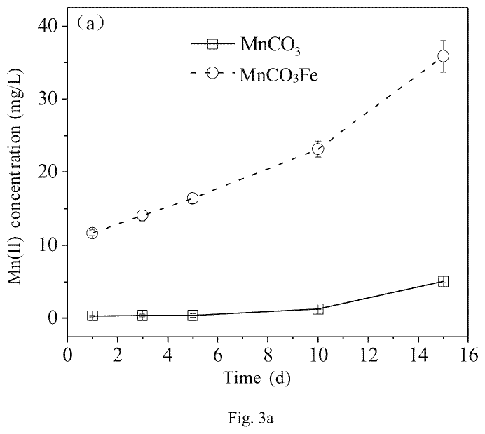

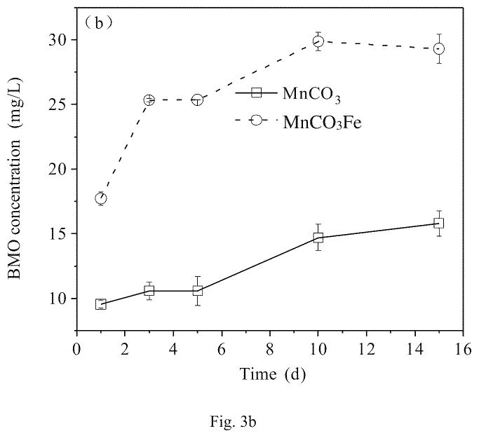

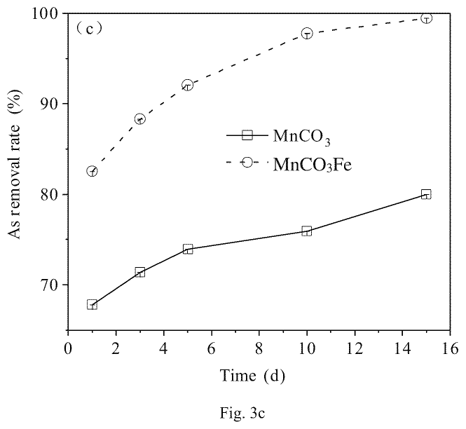

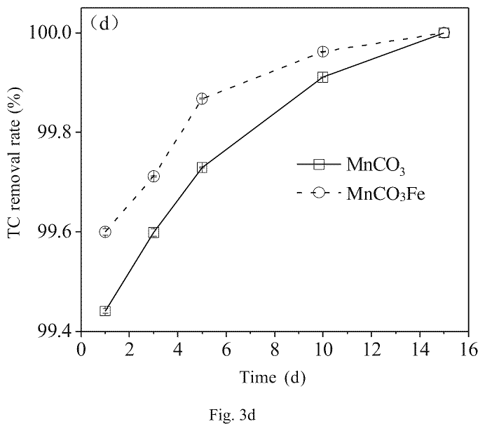

In Example 2, change curves of Mn(II) concentration, BMO concentration, As removal rate and TC removal rate over time were shown in FIGS. 3a-d. Referring to FIGS. 3a-d, a combined pollution system of As and the antibiotic were remediated under conditions in Example 2, and a concentration of soluble Mn(II) of MnCO₃Fe was higher than that of MnCO₃ group and reached a significant difference level. When the solid-phase manganese source was MnCO₃Fe, a concentration of BMO significantly increased, the total As removal rate reached 99.46%, and the total TC removal rate reached 100%. When the solid-phase manganese source was MnCO₃, the total As removal rate reached 80.01%, and the total TC removal rate reached 100%.

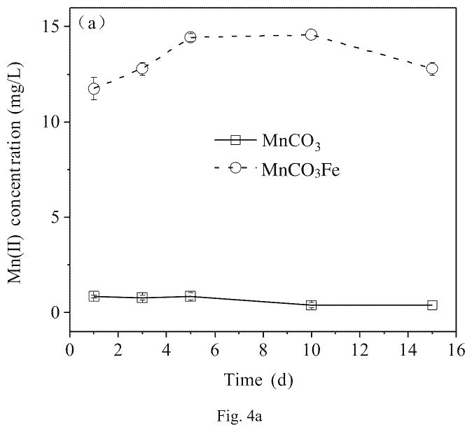

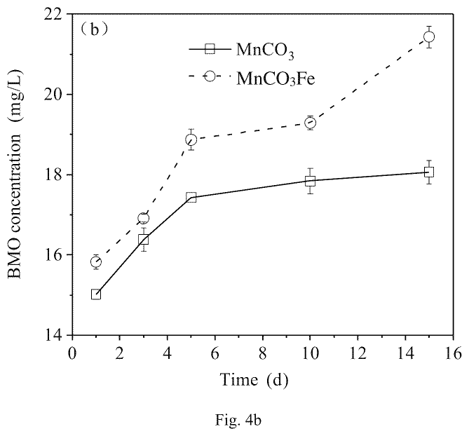

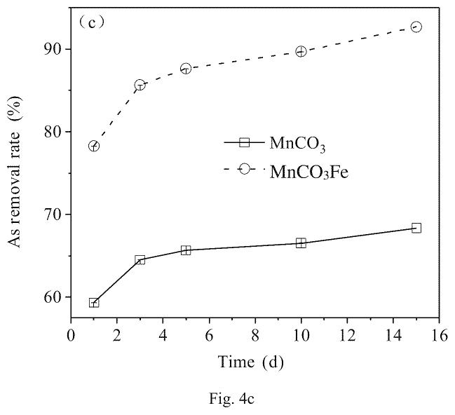

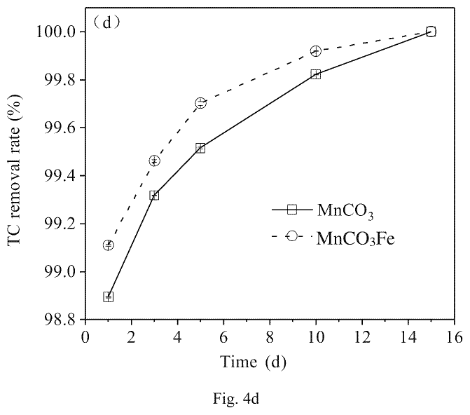

In Example 3, change curves of Mn(II) concentration, BMO concentration, As removal rate and TC removal rate over time were shown in FIGS. 4a-d. Referring to FIGS. 4a-d, a combined pollution system of As and the antibiotic were remediated under conditions in Example 3, and a concentration of soluble Mn(II) of MnCO₃Fe was higher than that of MnCO₃ group and reached a significant difference level. When the solid-phase manganese source was MnCO₃Fe, a concentration of BMO significantly increased, the total As removal rate reached 92.69%, and the total TC removal rate reached 100%. When the solid-phase manganese source was MnCO₃, the total As removal rate reached 68.35%, and the total TC removal rate reached 100%.

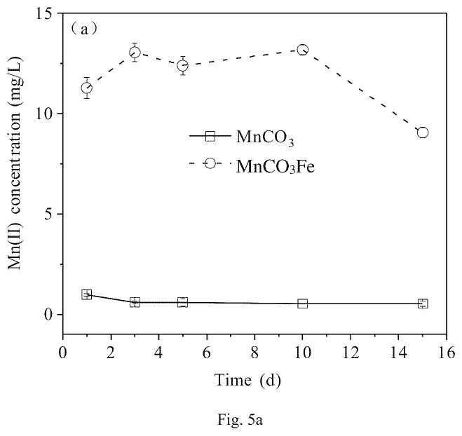

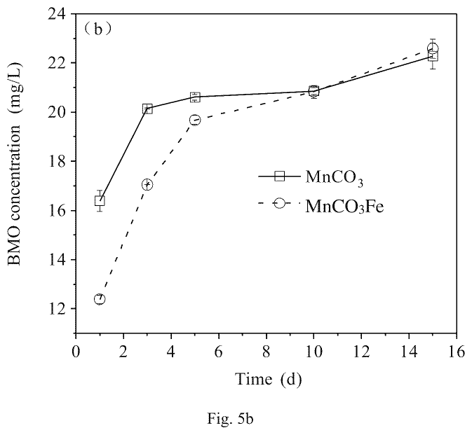

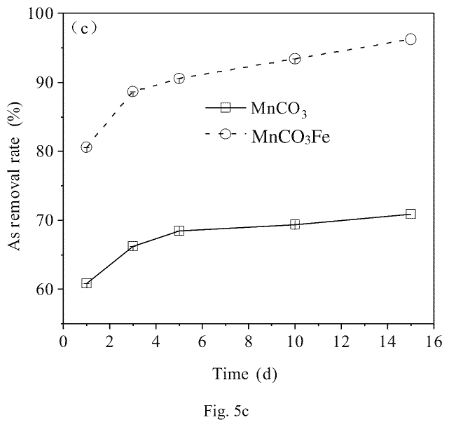

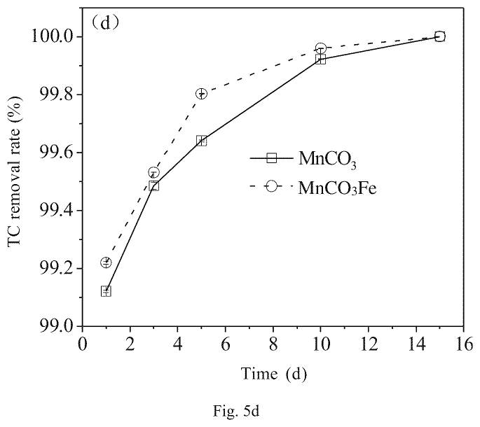

In Example 4, change curves of Mn(II) concentration, BMO concentration, As removal rate and TC removal rate over time were shown in FIGS. 5a-d. Referring to FIGS. 5a-d, a combined pollution system of As and the antibiotic were remediated under conditions in Example 4, and a concentration of soluble Mn(II) was higher than that of MnCO₃ group and reached a significant difference level. In the first five days of this reaction, concentrations of both groups rapidly increased and reached above 20 mg/L. When the solid-phase manganese source was MnCO₃Fe, the total As removal rate reached 96.25%, and the total TC removal rate reached 100%. When the solid-phase manganese source was MnCO₃, the total As removal rate reached 70.92%, and the total TC removal rate reached 100%.

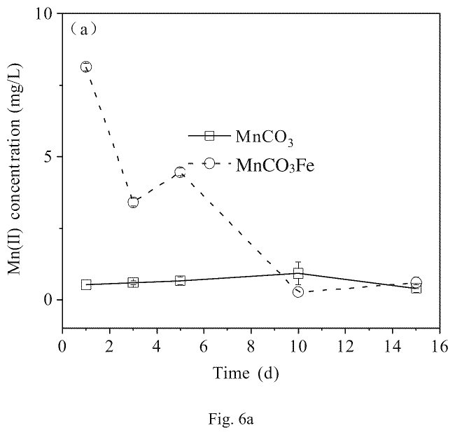

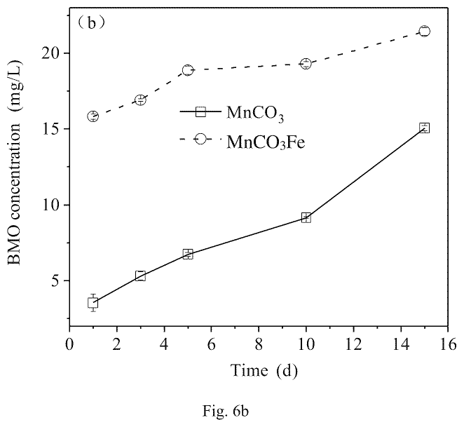

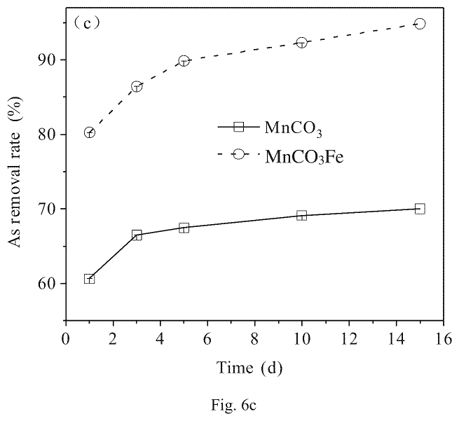

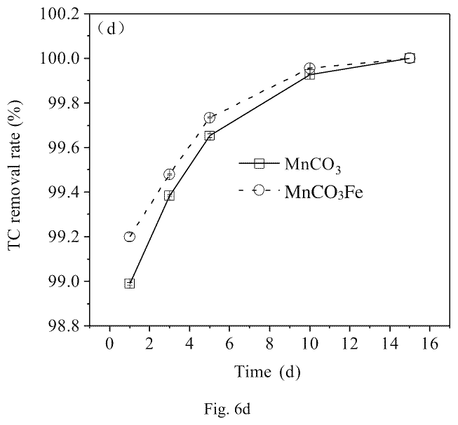

In Example 5, change curves of Mn(II) concentration, BMO concentration, As removal rate and TC removal rate over time were shown in FIGS. 6a-d. Referring to FIGS. 6a-d, a combined pollution system of As and the antibiotic were remediated under conditions in Example 5, and a concentration of soluble Mn(II) of MnCO₃Fe was higher than that of MnCO₃ group and reached a significant difference level. As reaction time passed by, a concentration of Mn(II) gradually decreased, while a concentration of BMO gradually increased. When the solid-phase manganese source was MnCO₃Fe, the total As removal rate reached 94.86%, and the total TC removal rate reached 100%. When the solid-phase manganese source was MnCO₃, the total As removal rate reached 70.02%, and the total TC removal rate reached 100%.

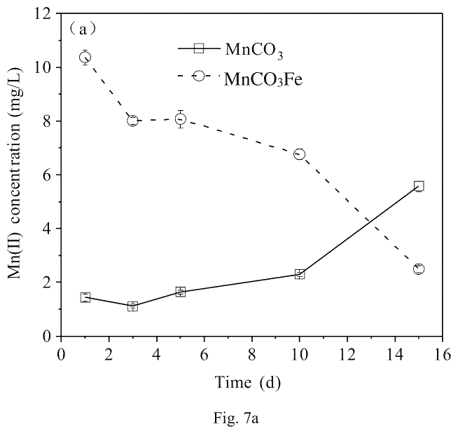

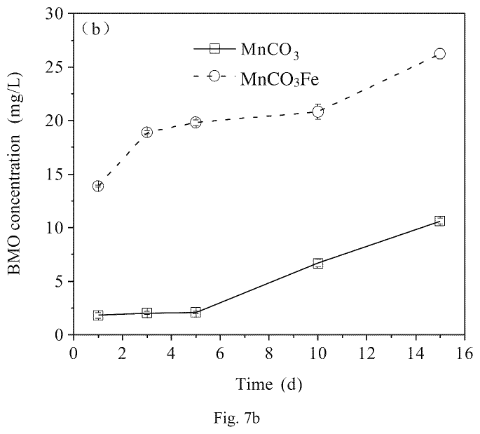

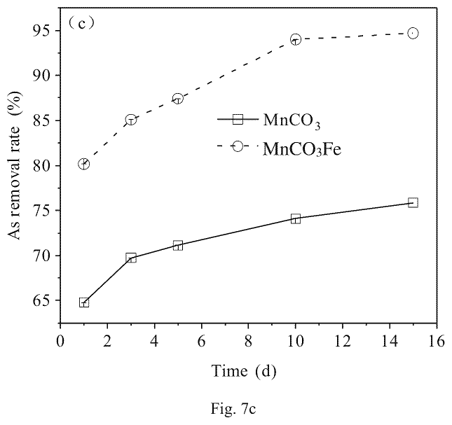

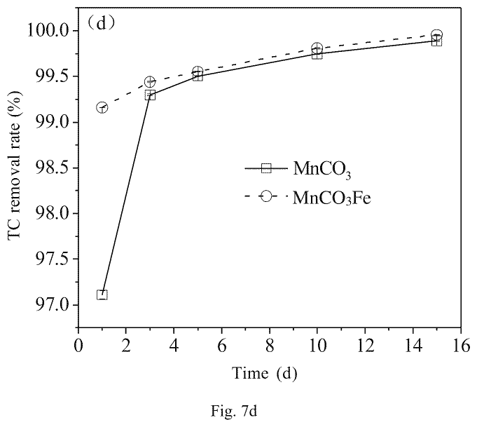

In Example 6, change curves of Mn(II) concentration, BMO concentration, As removal rate and TC removal rate over time were shown in FIGS. 7a-d. Referring to FIGS. 7a-d, a combined pollution system of As and the antibiotic were remediated under conditions in Example 6, and a concentration of soluble Mn(II) of MnCO₃Fe was higher than that of MnCO₃ group and reached a significant difference level. As reaction time passed by, a concentration of Mn(II) gradually decreased, while a concentration of BMO gradually increased. When the solid-phase manganese source was MnCO₃Fe, the total As removal rate reached 94.68%, and the total TC removal rate reached 100%. When the solid-phase manganese source was MnCO₃, the total As removal rate reached 75.85%, and the total TC removal rate reached 100%.

From the comparison of total As removal rates and total TC removal rates of MnCO₃ and MnCO₃Fe in Examples 1-6, the solid-phase manganese source of MnCO₃Fe has better remediation effects on pollution system of As and the antibiotic than that of MnCO₃.

It should be noted that the above embodiments are illustrative rather than limiting the present disclosure. Although the present disclosure is described in detail according to preferred embodiments, it should be understood by those skilled in the art that modifications and equivalent replacements of the technical solutions of the present disclosure without departing from the spirit and scope of the technical solutions of the present disclosure shall fall within the scope of the disclosure defined by the appended claims.

What is claimed is:

1. A method for preparing a manganese carbonate-supported ferrihydrite material, comprising:
(S1) dissolving manganese sulfate with water to obtain a manganese sulfate solution, and dissolving ammonium bicarbonate with water to obtain an ammonium bicarbonate solution;
(S2) adding ferrihydrite and dropwise adding the ammonium bicarbonate solution to the manganese sulfate solution followed by reaction under magnetic stirring to obtain a first precipitate; and
(S3) washing the first precipitate with distilled water followed by drying in a drying oven to obtain the manganese carbonate-supported ferrihydrite material.

2. The method of claim 1, wherein in step (S1), a concentration of the manganese sulfate solution is 0.5-1.2 mol/L; and a concentration of the ammonium bicarbonate solution is 1.5-2.3 mol/L.

3. The method of claim 1, wherein in step (S2), an addition amount of the ferrihydrite is 50-120 g/L.

4. The method of claim 1, wherein in step (S2), the ferrihydrite is prepared through steps of:
(S2.1) dissolving 40 g of ferric nitrate nonahydrate in 500 mL of water followed by magnetic stirring to obtain a ferric nitrate solution;
(S2.2) adding 310 mL of a 1 mol/L potassium hydroxide solution to the ferric nitrate solution at a speed of 100 mL/min to obtain a mixed solution;
(S2.3) adjusting the mixed solution to pH 7-8 with the 1 mol/L potassium hydroxide solution followed by stirring for 1 h to produce a supernatant and a second precipitate; and
(S2.4) washing the second precipitate with a 0.1 mol/L sodium chloride solution 2-3 times followed by centrifugation at 3000 r/min for 8-12 min and drying at 80° C. for 10-12 h to obtain the ferrihydrite.

5. The method of claim 1, wherein in step (S3), the first precipitate is washed 2-4 times successively with 90-100° C. distilled water and 20-30° C. distilled water; and the drying is performed at 80-90° C. in the drying oven for 14-20 h.

6. A manganese carbonate-supported ferrihydrite material prepared by the method of claim 1.

7. A method for treating a wastewater sample polluted by arsenic (As) and an antibiotic by using the manganese carbonate-supported ferrihydrite material of claim 6, comprising:
(a) inoculating Pseudomonas putida strain MnB1 with an accession number of ATCC 23483 into a culture medium A for culture to obtain a Pseudomonas putida suspension; and
(b) adding the Pseudomonas putida suspension into a culture medium B containing the manganese carbonate-supported ferrihydrite material followed by addition of 4-(2-hydroxyethyl) piperazine-1-ethanesulfonic acid (HEPES) and the wastewater sample in a clean bench to realize removal of As and the antibiotic.

8. The method of claim 7, wherein in step (a), the culture medium A comprises 0.3-0.8 g/L of yeast extract, 0.2-0.8 g/L of casein acid hydrolysate, 0.3-0.8 g/L of glucose, 0.1-0.4 g/L of calcium chloride, 0.1-0.6 g/L of magnesium sulfate, 1-5 mL/L of a trace element and 1-3 mL/L of ferric chloride, and a pH of the culture medium A is 7; and
the culture is performed through steps of:
inoculating the Pseudomonas putida strain MnB1 into the culture medium A with an inoculation amount of 3-5% by volume followed by culture under shaking and aerobic conditions at 25-35° C. for 1-2 days.

9. The method of claim 7, wherein in step (b), an addition amount of the Pseudomonas putida suspension is 3-5% by volume of the culture medium B; an addition amount of the manganese carbonate-supported ferrihydrite material is 0.1-2.0 g/L; the culture medium B comprises 0.1-0.4 g/L of ammonium ferrous sulfate, 0.1-0.4 g/L of sodium citrate, 0.05-0.2 g/L of yeast extract powder and 0.02-0.1 g/L of sodium pyrophosphate, and a pH of the culture medium B is 6-8; an initial concentration of As in the wastewater sample is 0.5-3 mg/L; and an initial concentration of the antibiotic in the wastewater sample is 10-40 mg/L.
